(19) United States
(12) Patent Application Publication
(10) Pub. No.: US 2010/0190809 A1
(43) Pub. Date: Jul. 29, 2010

(54) COMBINATION FORMULATIONS

(76) Inventors: Jody Firmin Voorspoels, Reningelst (BE); Eugeen Maria Jozef Jans, Meerhout (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/600,850

(22) PCT Filed: Jun. 25, 2008
(86) PCT No.: PCT/EP08/58077

§ 371 Date: Nov. 19, 2009

(30) Foreign Application Priority Data
Jun. 25, 2007 (EP) 07110998.7

(51) Int. Cl. A61K 31/505

(52) U.S. Cl. 514/272

(57) ABSTRACT

This invention relates to solid oral dosage forms of the HIV inhibitors containing a combination of TMC114 and TMC125.

### FIELD OF THE INVENTION

This invention relates to solid oral dosage forms of the HIV inhibitors containing a combination of TMC114 and TMC125.

### BACKGROUND OF THE INVENTION

The treatment of Human Immunodeficiency Virus (HIV) infection, known as cause of the acquired immunodeficiency syndrome (AIDS), remains a major medical challenge. HIV is able to evade immunological pressure, to adapt to a variety of cell types and growth conditions and to develop resistance against currently available drug therapies. The latter include nucleoside reverse transcriptase inhibitors (NRTIs), non-nucleoside reverse transcriptase inhibitors (NNRTIs), nucleotide reverse transcriptase inhibitors (NtRTIs), HIV-protease inhibitors (PIs), fusion inhibitors, and the more recent CCRS and integrase inhibitors.

Although effective in suppressing HIV, each of these drugs, when used alone, is confronted with the emergence of resistant mutants. This led to the introduction of combination therapy of several anti-HIV agents usually having a different activity profile. In particular the introduction of “HAART” (Highly Active Anti-Retroviral Therapy) resulted in a remarkable improvement in anti-HIV therapy, leading to a large reduction in HIV-associated morbity and mortality. However, none of the currently available combination therapies is capable of completely eradicating HIV. Even HAART may face the emergence of resistance, often due to non-adherence and non-persistence with antiretroviral therapy. In these cases HAART can be made effective again by replacing one of its components by one of another class. If applied correctly, treatment with HAART combinations can suppress the virus for many years, up to decades, to a level where it no longer can cause the outbreak of AIDS.

Because of their pharmacokinetic properties and the need to keep plasma levels above a minimum level, currently used anti-HIV drugs require frequent administration of relatively high doses. The number and/or volume of dosage forms that need to be administered are commonly referred to as the “pill burden”. A high pill burden is undesirable for many reasons, such as the frequency of intake, often combined with the inconvenience of having to swallow large dosage forms, as well as the need to store and transport a large number or volume of pills. A high pill burden increases the risk of patients not taking their entire dose, thereby failing to comply with the prescribed dosage regimen. As well as reducing the effectiveness of the treatment, this also leads to the emergence of viral resistance. The problems associated with a high pill burden are multiplied where a patient must take a combination of different anti-HIV agents.

The complex dosing regimens of HAART or other dosing regimens can be simplified by the application of combination dosage forms comprising two or more anti-HIV components. These could take the form of fixed dose combinations, e.g. tablets comprising predetermined doses of two or more anti-HIV agents. Most HIV inhibitors however need to be administered at relatively high doses so that often two or more doses need to be administered at once in order to reach the required quantity. Combination dosage forms would become so large that their intake becomes inconvenient or even impossible. For reasons of convenience, a solid oral dosage form should not exceed about 1400 mg, and preferably should not exceed about 1300 mg or about 1200 mg. Providing dosage forms of relatively small size contributes to the convenience of intake and therefore also helps to overcome the problems of pill burden.

Therefore, it would be desirable to provide HIV inhibitory therapy that reduces pill burden in that it involves the administration of dosage forms of a practical size and additionally does not require frequent dosing.

One class of HIV drugs that is often used in HAART is that of the NNRTIs of which a number are currently on the market and several others are in various stages of development. One such NNRTI is the compound 4-[[6-amino-5-bromo-2-[(4-cyanophenyl)amino]-4-pyrimidinyl]oxy]-3,5-dimethylbenzonitrile, also referred to as etravirine, R 165335, or in particular as TMC125, and is currently on the market in a number of countries under the tradename Intelence™. TMC125 can be represented by the formula (I):

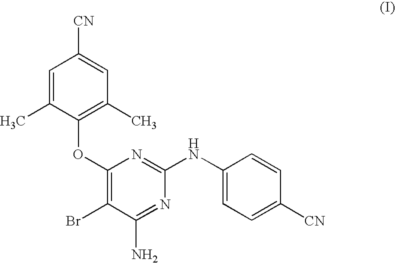

This compound, its properties, a number of synthetic approaches for its preparation, as well as standard pharmaceutical formulations, have been described in WO 00/27825. TMC125 not only shows pronounced activity against wild type HIV, but also against HIV strains harboring resistance-inducing mutations.

TMC125 is very insoluble in aqueous media and therefore has low bioavailability. WO 01/23362 and WO 01/22938 disclose solid dispersions of this compound in water-soluble polymers offering improved bioavailability, especially when in the form of powders prepared by spray drying. An improved spray dried formulation has been disclosed in WO 2007/141308 (published 13 Dec. 2007). Current tablet formulations of TMC125 are based on a solid dispersion of TMC125 in hydroxypropyl methylcellulose (HPMC) obtained by spray drying. The spray-dried powder is mixed with further ingredients and compressed to a tablet dosage form. The spray-dried solid dispersion however is difficult to compress requiring the addition of binders such as one or more of those mentioned hereinafter, in particular one or more of microcrystalline cellulose and lactose.

The current dosing regimen of TMC125 is 200 mg twice a day (b.i.d.), administered as two tablets each containing 100 mg, to be taken in at once, preferably two in the morning and two at the end of the day. Because these quantity requirements and the fact that TMC125 is dispersed in a relatively large quantity of water-soluble polymer, oral dosage forms of this drug are inevitably large in size.

Another class of HIV drugs that is used in HAART is that of the PIs amongst which is TMC114 (darunavir), approved in the U.S., the E.U. and a number of other countries and available under the tradename Prezista™. TMC114 used in the form of darunavir monoethanolate, has the following chemical name: (1S,2R,3R,3aS,6aR)-{3-[(4-aminobenzenesulfonyl)isobutyl-amino]-1-benzyl-2-hydroxypropyl}carbamic acid hexahydrofuro[2,3-b]furan-3-yl ester monoethanolate. Its molecular formula is C₂₂H₃₇N₃O₂S.C₂H₅OH, with a molecular weight of 593.73, and the following chemical structure:

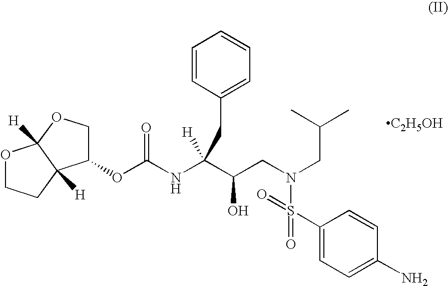

TMC114 as well as processes for its preparation have been disclosed in EP 715618, WO 99/67417, U.S. Pat. No. 6,248,775, and in Bioorganic and Chemistry Letters, Vol. 8, pp. 687-690, 1998, “Potent HIV protease inhibitors incorporating high-affinity P₂-ligands and (R) (hydroxyethylamino)sulfonamide isostere”.

TMC114 is a welcome addition to the class of PI drugs because of its pronounced activity against wild type virus, but in particular against many mutated variants posing a large barrier against the development of resistance.

Current HAART combinations comprise two NRTIs combined with an NNRTI or two NRTIs with a PI. In certain circumstances it is desirable to add an NNRTI to the latter combination in order to increase the barrier for drug-escaping mutations, in particular where such combination is used in so-called salvage therapy. Both TMC114 and TMC125 have a high genetic barrier against drug-escaping mutations so that a combination of these two drugs is expected to create an almost insurmountable barrier. A combination of these two drugs may find use as well in experienced, in less experienced or even in so called drug-naïve patients and therefore provides additional therapeutic options for HIV infected patients.

In order to reduce pill-burden it would be desirable to combine TMC114 and TMC125 in one and the same dosage form. Because TMC125 dosage forms are inevitably large in size, combination dosage forms with other anti-HIV drugs would take a size that surpasses the convenience barrier.

Indeed, the TMC125 tablets currently on the market in a number of countries contain 100 mg of active ingredient and have a total weight of 800 mg per tablet, are oval shaped with following dimensions: length 19 mm, width 9.5 mm and a radius of 7.33 mm. The TMC114 tablets currently on the market contain 300 mg weight equivalents of active ingredient (corresponding to 325.25 mg of TMC114 monoethanolate), having a total weight of 625.2 mg per tablet, and are oval shaped with following dimensions: length 17.3 mm, width 8.64 mm and a radius of 7.78 mm. A combination tablet therefore would weigh 1425.2 mg and would be large in size exceeding the convenience size limit so that patients will have difficulties, perceived or real, in taking in such large tablets. This contributes to pill burden, the initial problem combination tablets were aimed to deal with.

Thus in one aspect, the present invention is aimed at providing more compact dosage forms that contain both TMC125 and TMC114, allowing a more convenient and acceptable size of such dosage forms. The present invention is based on the unexpected finding that the HIV inhibitor TMC114 functions as a binder, a property unique to an active ingredient, and therefore can take over the role of such agent. This allows for the use of less binder resulting in more compact dosage forms so that TMC114 can be combined with TMC125 without exceeding the size barrier of an acceptable oral dosage form.

The dosage forms of this invention provide anti-HIV therapy involving the administration of combined dosage forms of acceptable size, thereby requiring less frequent dosing. Hence, present dosage forms are beneficial in terms of pill burden and drug compliance of the patient.

### SUMMARY OF THE INVENTION

The present invention is based on the finding that TMC114 acts as a binder in compressed solid pharmaceutical dosage forms such as for oral administration.

Thus, the present invention relates to a solid pharmaceutical oral dosage form comprising:
(a) from about 15 mg to about 200 mg of TMC125, or from about 25 mg to about 150 mg of TMC125, or from about 50 to about 150 mg of TMC125, or from about 80 to about 120 mg of TMC125, dispersed in a solid dispersion with a water-soluble polymer;
(b) from about 50 to about 600 mg, or from about 50 mg to about 500 mg of free-form equivalent TMC114; or from about 250 mg to about 450 mg of free-form equivalent of TMC114, or from about 250 mg to about 350 mg, of free-form equivalent of TMC114;
(c) from about 200 mg to about 400 mg of a carrier;
   the total weight of the dosage form not exceeding 1300 mg.

In one embodiment the TMC125 in the dosage forms of the invention is dispersed in from about 100 mg to about 500 mg, or from about 200 mg to about 400 mg, for example 300 mg of a water-soluble polymer.

Further embodiments comprise solid pharmaceutical oral dosage forms as described above or hereinafter wherein the solid dispersion of TMC125 comprises from about 10 mg to about 100 mg, or from about 20 mg to about 80 mg, or from about 30 mg to about 70 mg, or from about 40 mg to about 60 mg of microcrystalline cellulose.

In one embodiment the invention concerns a solid pharmaceutical oral dosage form comprising
(a) about 100 mg of TMC125 dispersed in a solid dispersion with a water-soluble polymer;
(b) about 300 mg free-form equivalent of TMC114, or in particular about 325 mg of TMC114 ethanolate; or about 400 mg free-form equivalent of TMC114, or in particular about 434 mg of TMC114 ethanolate;
(c) from about 200 mg to about 400 mg of a carrier;
   the total weight of the dosage form not exceeding 1300 mg.

Further embodiments comprise solid pharmaceutical oral dosage forms as described above or hereinafter wherein the solid dispersion of TMC125 comprises from about 50 mg of microcrystalline cellulose.

### DESCRIPTION OF THE INVENTION

As mentioned above, TMC114 acts as a binder. An inactive ingredient (excipient) termed a “binder” is added to help hold the tablet together and give it strength. A wide variety of binders may be used, some common ones including lactose, dibasic calcium phosphate, sucrose, corn (maize) starch, microcrystalline cellulose and modified cellulose (for example hydroxymethyl cellulose). Other such materials are silicon dioxide, titanium dioxide, alumina, talc, kaolin, powdered cellulose, as well as soluble materials such as mannitol, urea, sucrose, lactose, dextrose, sodium chloride, and sorbitol. Such agents may sometimes also be referred to as “fillers”.

The present invention provides a solid oral dosage form of TMC114 and TMC125 of a size below the combined size of both dosage forms. The size of the dosage forms of the invention, i.e. the total weight of the dosage forms, should be below a limit of convenience which is below the size at which a number of patients starts having difficulty taking in the dosage form. The total weight of the dosage forms of the invention may be below about 1300 mg, and in particular below about 1200 mg. In particular embodiments the total weight of the dosage forms in accordance with the present invention is below about 1100 mg, or is below about 1000 mg. Or alternatively, the volume of the oral dosage forms of the invention may be below about 1.3 cm³ (in particular below about 1.300 cm³ or below about 1300 mm³) and in particular below about 1.200 cm³ (in particular below about 1.200 cm³ or below about 1200 mm³) In particular embodiments the volume of the dosage forms in accordance with the present invention is below about 1.100 cm³, or is below about 1.000 cm³.

The solid oral dosage forms of the present invention preferably are tablets.

The dosage forms of the invention contain the quantities of the active ingredients mentioned above. For certain patient groups certain specific quantity ranges may be recommended. For paediatric applications, lower quantities of the active ingredients may be used. In such instance, the dosage forms of the invention contain from about 15 to about 50 mg, or from about 20 to about 30 mg, in particular about 25 mg of TMC125 per unit of the dosage form, wherein the TMC125 is dispersed in a solid dispersion with a water-soluble polymer. Such dosage forms for paediatric applications may contain from about 50 to about 200 mg, or from about 50 mg to about 150 mg, in particular about 75 mg of TMC114 of free-form equivalent TMC114 per unit of the dosage form. The total weight of the dosage forms for paediatric applications may vary, but in particular is below about 433 mg, and more in particular below about 400 mg, or below about 367 mg, or below about 333 mg. For application in adults, higher quantities of the active ingredients may be used. In such instance, the dosage forms of the invention contain from about 50 to about 200 mg, in particular from about 75 mg to about 150 mg, more in particular 90 mg to about 120 mg, for example about 100 mg, of TMC125 per unit of the dosage form. Such dosage forms for application in adults may contain from about 150 to about 600 mg, or from about 200 mg to about 400 mg, in particular about 300 mg of free-form equivalent TMC114 per unit of the dosage form.

In an alternative embodiment, for paediatric applications, the quantities or ranges of quantities of the active ingredients TMC125 and TMC114 in the dosage forms of the invention, or the total weights or volumes of the dosage forms, or both, may be those mentioned in relation to the application for adults, divided by 3. In another alternative embodiment, for applications in adolescents, the quantities of the active ingredients TMC125 and TMC114 in the dosage forms of the invention may be those mentioned in relation to the application for adults, divided by 2, by 1.7, by 1.5, or by 1.33.

The weight/weight ratio TMC125:TMC114 may vary, but in one embodiment it is in the range from about 1:1 to about 1:5, or from about 1:2 to about 1:4; in particular said ratio may be about 1:3.

The active ingredients used in the formulations of the invention are the NNRTI TMC125 and the PI TMC114. Both can be used in base form or as a pharmaceutically acceptable addition salt form, in particular as an acid addition salt form, or as a pharmaceutically acceptable solvate. The pharmaceutically acceptable addition salts are meant to comprise the therapeutically active non-toxic salt forms. The acid addition salt forms can be obtained by treating the base form with appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propane-tricarboxylic, methanesulfonic, ethanesulfonic, benzenesulfonic, 4-methylbenzene-sulfonic, cyclohexanesulfamic, 2-hydroxybenzoic, 4-amino-2-hydroxybenzoic and the like acids.

The term pharmaceutically acceptable solvate comprises the hydrates and the solvent addition forms that the HIV inhibitors TMC125 and TMC114 can form. Examples of such forms are e.g. hydrates, alcoholates, e.g. methanolates, ethanolates and propanolates, and the like.

As used herein, the term “TMC125” is meant to comprise the base form, any pharmaceutically acceptable acid addition salt thereof, as well as any pharmaceutically acceptable solvate thereof. The pharmaceutically acceptable addition salts as mentioned hereinabove comprise the therapeutically active non-toxic acid addition salt forms, which TMC125 is able to form. In one embodiment the term “TMC125” is meant to comprise the base form, as well as any pharmaceutically acceptable acid addition salt thereof. Particular acid addition salts are the hydrohalic salts, e.g. the hydrochloride or hydrobromide salt.

As used herein, the term “TMC114” is meant to comprise the base form, any pharmaceutically acceptable acid addition salt thereof, as well as any pharmaceutically acceptable solvate thereof. The pharmaceutically acceptable addition salts as mentioned hereinabove comprise the therapeutically active non-toxic acid addition salt forms, which TMC114 is able to form. In one embodiment the term “TMC114” is meant to comprise the base form, as well as any pharmaceutically acceptable solvate thereof. Particular solvates are the ethanolate, e.g. the monoethanolate.

TMC125 preferably is used in free form, also referred to as base form, TMC114 as monoethanolate.

As used herein the term “free-form equivalent TMC114” refers to that quantity of TMC114, whether present in free form (or base form), or as salt or solvate, that corresponds to a given quantity of TMC114 in free form. For example 325 mg of TMC114 monoethanolate corresponds to 300 mg of free-form equivalent TMC114.

The TMC125 in the dosage forms of the invention is present in the form of a solid dispersion in a water-soluble polymer. Different types of solid dispersions exist. One type of solid dispersion is where the pharmaceutical agent is molecularly dispersed, substantially homogeneously, throughout the polymer. This is generally described as a “solid solution”. Another type of solid dispersion is where there are islands or clusters of crystalline or semi-crystalline pharmaceutical agent dispersed throughout the polymer. A further type of solid dispersion is where there are islands or clusters of pharmaceutical agent in amorphous form dispersed throughout the polymer. There may also be solid dispersions comprising mixtures of two or more of the above types, for example a solid solution with areas where the pharmaceutical agent is crystalline or semi-crystalline, or where there are islands or clusters of the agent in amorphous form. All these types will be commonly designated hereinafter as “solid dispersions”.

The TMC125 is dispersed in a water-soluble polymer present in a quantity of about 100 to about 500 mg, in particular about 200 to about 400 mg, more in particular about 250 to about 350 mg, for example about 300 mg, of water-soluble polymer per dosage unit. The weight/weight ratio of TMC125 to the water-soluble polymer may be in the range of about 1:1 to about 1:10, in particular of about 1:1 to about 1:5, more in particular of about 1:2 to about 1:4, for example said ratio may be about 1:3. If desired, other materials may be added when preparing the solid dispersion.

In one embodiment, there is provided a solid pharmaceutical oral dosage form comprising:
(a) from about 1% to about 15% of TMC125, or from about 2% to about 12% of TMC125, or from about 4% to about 12% of TMC125, or from about 6% to about 9% of TMC125, dispersed in a solid dispersion with a water-soluble polymer;
(b) from about 4% to about 45%, or from about 5% to about 40% of free-form equivalent TMC114; or from about 20% to about 35% of free-form equivalent TMC114, or from about 20% to about 27% of free-form equivalent of TMC114;
(c) from about 15% to about 30% of a carrier;
   the total weight of the dosage form not exceeding 1300 mg and all above percentages being by weight relative to the total weight of the dosage form. In another embodiment, the above percentages are multiplied by 1.0833 and the total weight of the dosage form does not exceed 1200 mg. In still another embodiment, the above percentages are multiplied by 1.18182 and the total weight of the dosage form does not exceed 1100 mg.

In one embodiment, there is provided a solid pharmaceutical oral dosage form comprising:
(a) about 7.7% of TMC125 dispersed in a solid dispersion with a water-soluble polymer;
(b) about 23% free-form equivalent of TMC114, or in particular about 25% of TMC114 monoethanolate; or about 30% free-form equivalent of TMC114, or in particular about 33% of TMC114 monoethanolate;
(c) from about 15% to about 30% of a carrier;
   the total weight of the dosage form not exceeding 1300 mg and all above percentages being by weight relative to the total weight of the dosage form. In another embodiment, the above percentages are multiplied by 1.0833 and the total weight of the dosage form does not exceed 1200 mg. In still another embodiment, the above percentages are multiplied by 1.18182 and the total weight of the dosage form does not exceed 1100 mg.

Particular embodiments are those wherein in the dosage forms of the previous two paragraphs the TMC125 is dispersed in from about 8% to about 40%, or from about 15% to about 30%, or in particular in about 23% of a water-soluble polymer. Further embodiments comprise solid pharmaceutical oral dosage forms as described above or hereinafter wherein the solid dispersion of TMC125 comprises from about 1% to about 7.5%, or from about 1.5% to about 6%, or from about 2% to about 5%, or from about 3% to about 4.5%, e.g. about 3.85% of microcrystalline cellulose. The foregoing percentages being for the instance where the total weight of the dosage form does not exceed 1300 mg and wherein all above percentages are by weight relative to the total weight of the dosage form. In other embodiments, the above percentages are multiplied by 1.0833 and the total weight of the dosage form does not exceed 1200 mg. In still another embodiment, the above percentages are multiplied by 1.18182 and the total weight of the dosage form does not exceed 1100 mg.

An ingredient that may be added to the spray mixture is microcrystalline cellulose (MCC) resulting in a powder of increased density thereby improving properties such as flowability.

The solid dispersion of TMC125 may be prepared using standard procedures but preferably is prepared by spray drying. In this procedure, a feed mixture of a solution of a water-soluble polymer and TMC125, optionally in admixture with microcrystalline cellulose and other ingredients, is spray-dried to form a solid dispersion of the pharmaceutical agent and the polymer by introducing the feed mixture as droplets into a spray-drying chamber via an atomizing means. Typically a heated drying gas is used to assist removal of the solvent. The TMC125 active agent in the spray-dried formulations solid dispersion is in a highly amorphous state, with little crystallinity, thereby improving bioavailabilty.

The solid dispersion of TMC125 typically comprises particles having an average effective particle size in the range of from about 10 μm to about 150 μm, or about 15 μm to about 100 μm, particularly about 20 μm to about 80 μm, or 30 μm to about 50 μm, preferably about 40 μm. As used herein, the term average effective particle size has its conventional meaning as known to the person skilled in the art and can be measured by art-known particle size measuring techniques such as, for example, sedimentation field flow fractionation, photon correlation spectroscopy, laser diffraction or disk centrifugation. The average effective particle sizes mentioned herein may be related to weight distributions of the particles. In that instance, by “an average effective particle size of about 150 μm” it is meant that at least 50% of the weight of the particles has a particle size of less than the effective average of 150 μm, and the same applies to the other effective particle sizes mentioned. In a similar manner, the average effective particle sizes may be related to volume distributions of the particles but usually this will result in the same or about the same value for the average effective particle size.

The so-called “span” of the particles produced by the process of the invention may be lower than about 3, in particular lower than about 2.5, preferably the span is about 2. Usually the span will not be lower than about 1. As used herein the term “span” is defined by the formula (D₉₀−D₁₀)/D₅₀ wherein D₉₀ is the particle diameter corresponding to the diameter of particles that make up 90% of the total weight of all particles of equal or smaller diameter and wherein D₅₀ and D₁₀ are the diameters for 50 respectively 10% of the total weight of all particles.

The amount of TMC125 in the spray dried product may be in the range from about 10% to about 50%, in particular about 15% to about 40%, or about 20% to about 30% or about 20% to about 25%, by weight relative to the total weight of the spray dried product comprising TMC125, water-soluble polymer, optional MCC and further optional excipients. The amount of TMC125 in the feed mixture can be calculated based on these percentages and on the amount of solvent used.

The microcrystalline cellulose (MCC) that can be added to the mixture for spray-drying has an average particle size, which is selected such that when mixed into the solution of pharmaceutical agent and water-soluble polymer, the resulting feed mixture is able to pass through the atomizing means into the spray-drying chamber without clogging or blocking the atomizer. As such, the size of the MCC is limited by the particular size of the atomizing means provided on the spray-drying chamber. For example, where the atomizing means is a nozzle, the size of the nozzle bore will affect the size range of the MCC that may be used. The average particle size of the MCC may be in the range of from 5 μm to 50 μm, in particular from 10 μm to 30 μm, e.g. about 20 μm.

Microcrystalline cellulose that can be used comprises the Avicel™ series of products available from FMC BioPolymer, in particular Avicel PH 1050 (20 μm), Avicel PH 101® (50 μm), Avicel PH 301® (50 μm);

the microcrystalline cellulose products available from JRS Pharma, in particular Vivapur® 105 (20 μm), Vivapur® 101 (50 μm), Emcocel® SP 15 (15 μm), Emcocel® 50M 105 (50 μm), Prosolv® SMCC 50 (50 μm);
the microcrystalline cellulose products available from DMV, in particular Pharmacel® 105 (20 μm), Pharmacel®101 (50 μm); the microcrystalline cellulose products available from Blanver, in particular Tabulose (Microcel)®101 (50 μm), Tabulose (Microcel)®103 (50 μm);
the microcrystalline cellulose products available from Asahi Kasei Corporation, such as Ceolus® PH-F20JP (20 μm), Ceolus® PH-101 (50 μm), Ceolus® PH-301 (50 μm), Ceolus® KG-802 (50 μm).

A particularly preferred microcrystalline cellulose is Avicel PH 1050 (20 μm).

The amount of MCC in the spray dried product may be in the range from about 0% to about 25%, in particular from about 5% to about 20%, or from about 10% to about 15% or about 10% to about 12.5%, by weight relative to the total weight of the spray dried product comprising TMC125, water-soluble polymer, MCC and optional excipients. The weight ratio of the amounts of MCC to TMC125 in the spray dried product can be calculated based on these percentages and in particular may be in the range of from about 2:1 to about 1:7, in particular from about 1:1 to 1:5, preferably about 1:2. The amount of MCC in the feed mixture can be calculated based on these percentages and on the amount of solvent used. In view of the desirability of keeping the concentration of pharmaceutical agent in the resulting solid pharmaceutical composition as high as possible, the amount of MCC in the feed mixture is preferably kept as low as possible.

Polymers suitable for use in the process of this invention are pharmaceutically acceptable, water-soluble and substantially unreactive towards the pharmaceutical agent. Preferably, the polymer is water-soluble. Suitable polymers include cellulosic polymers, such as methyl cellulose, ethyl cellulose, hydroxymethyl cellulose, hydroxyl-ethyl cellulose, hydroxypropyl cellulose, hydroxybutyl cellulose, hydroxyethylmethyl cellulose, hydroxypropylmethyl cellulose (HPMC), e.g. HPMC 2910, carboxymethyl cellulose, hydroxypropylmethyl cellulose phthalate (HPMCP), e.g. HP 50, hydroxylpropylmethylcellulose acetate succinate (HPMCAS), cellulose acetate trimellitate (CAT), hydroxypropylcellulose acetate phthalate (HPCAP), hydroxy-propylmethyl cellulose acetate phthalate (HPMCAP), methylcellulose acetate phthalate (MCAP) and mixtures thereof such as a mixture of hydroxypropyl cellulose and ethyl cellulose. Suitable polymers also include polyvinyl pyrrolidone, copolyvidone, which is polyvinyl pyrrolidone copolymerised with vinyl acetate, and aminoalkyl methacrylate copolymers, such as Eudragit E® 100 (Röhm GmbH, Germany).

Preferably, the polymer is hydroxypropylmethyl cellulose (HPMC), polyvinyl pyrrolidone or copolyvidone. A particular hydroxypropylmethyl cellulose is HPMC 2910 5 mPa·s. A particular polyvinyl pyrrolidone is PVP K29-32, PVP K12, K30, K90, and a particular copolyvidone is PVP-co-VA64.

In one embodiment, the water-soluble polymer has a molecular weight in the range 500 D to 2 MD. The water-soluble polymer may have an apparent viscosity of 1 to 15,000 mPa·s, or of Ito 5000 mPa·s, or of Ito 700 mPa·s, or of Ito 100 mPa·s when in a 2% (w/v) aqueous solution at 20° C.

Particular hydroxyalkyl alkylcelluloses include hydroxyethyl methylcellulose and hydroxypropyl methylcellulose (or HPMC, e.g. HPMC 2910 15 mPa·s; HPMC 2910 5 mPa·s). Particular vinylpyrrolidones include PVP K29-32, PVP K90.

The HPMC that can be used may contain sufficient hydroxypropyl and methoxy groups to render it water-soluble. HPMC having a methoxy degree of substitution from about 0.8 to about 2.5 and a hydroxypropyl molar substitution from about 0.05 to about 3.0 are generally water-soluble. Methoxy degree of substitution refers to the average number of methyl ether groups present per anhydroglucose unit of the cellulose molecule. Hydroxypropyl molar substitution refers to the average number of moles of propylene oxide that have reacted with each anhydroglucose unit of the cellulose molecule. A preferred HPMC is hypromellose 2910 15 mPa·s or hypromellose 2910 5 mPa·s, especially hypromellose 2910 15 mPa·s. Hydroxypropyl methylcellulose is the United States Adopted Name for hypromellose (see Martindale, The Extra Pharmacopoeia, 29th edition, page 1435). In the four digit number “2910”, the first two digits represent the approximate percentage of methoxy groups and the third and fourth digits the approximate percentage composition of hydroxypropoxyl groups; 15 mPa·s or 5 mPa·s is a value indicative of the apparent viscosity of a 2% aqueous solution at 20° C.

The amount of water-soluble polymer in the spray dried product may be in the range from about 30% to about 75%, in particular about 40% to about 75%, or about 50% to about 75% or about 60% to about 70%, by weight relative to the total weight of the spray dried product comprising TMC125, water-soluble polymer, MCC and optional excipients. The amount of water-soluble polymer in the feed mixture can be calculated based on these percentages and on the amount of solvent used.

The weight:weight ratio of water-soluble polymer to TMC125 may be in the range from 10:1 to 1:10, in particular from 10:1 to 1:1, more in particular from 5:1 to 1:1, preferably from about 3:1 to 1:1, e.g. a ratio of about 3:1 or of about 2:1. It may be desirable to reduce the amount of polymer in relation to the pharmaceutical agent in order to maximize the amount of pharmaceutical agent in the resulting pharmaceutical composition. This is the case were larger quantities of the other ingredients are used, for example were relatively large quantities of TMC114 or TMC125, or both, are used.

The solvent used in the method of the present invention may be any solvent, which is inert with respect to TMC125 and which is able to dissolve TMC125 and the water-soluble polymer. In case MCC is added, the solvent should not dissolve the MCC. Suitable solvents include acetone, tetrahydrofuran (THF), dichloromethane, ethanol (anhydrous or aqueous), methanol and combinations thereof. Where the polymer is HPMC, the solvent preferably is a mixture of dichloromethane and ethanol, more preferably a mixture of dichloromethane and ethanol, the latter in particular being anhydrous ethanol, in a 9:1 ratio by weight. Where the polymer is polyvinyl pyrrolidone or copolyvidone, the solvent is preferably acetone.

Examples of feed mixtures that can be used in the process of the invention are those comprising:
(i) 200 mg TMC125, 200 mg HPMC 2910 5 mPa·s, 100 mg microcrystalline cellulose (Avicel PH 1050) in 14.57 g dichloromethane extra pure and 1.619 g ethanol 96% (v/v);
(ii) 200 mg TMC125, 400 mg HPMC 2910 5 mPa·s, 100 mg microcrystalline cellulose (Avicel PH 1050) in 14.57 g dichloromethane extra pure and 1.619 g ethanol 96% (v/v);
(iii) 200 mg TMC125, 600 mg HPMC 2910 5 mPa·s, 100 mg microcrystalline cellulose (Avicel PH 1050) in 14.57 g dichloromethane extra pure and 1.619 g ethanol 96% (v/v);
(iv) 222 mg TMC125, 667 mg HPMC 2910 5 mPa·s, 111 mg microcrystalline cellulose (Avicel PH 1050) in 16.19 g dichloromethane extra pure and 1.8 g ethanol absolute (100%).

The above feed mixtures can be scaled up by multiplying the quantities mentioned by a factor that is in the range of about 1 to about 10⁵. In lab scale production the quantities may be multiplied by a factor in the range of about 1 to about 1000. For medium or large scale production this factor may be in the range of about 500 to about 10⁵, e.g. about 10³, about 2×10³, about 5×10³ or about 10⁴.

Feed mixtures (i)-(iv) can also be used without MCC. Feed mixtures (i)-(iv), with or without MCC, can be scaled up by multiplying by the factors mentioned above.

The solvent is removed from the droplets of the feed mixture by the spray-drying step. Preferably the solvent is volatile, with a boiling point of 150° C. or less, preferably 100° C. or less. The solvent should be substantially completely removed from the droplets of the feed mixture during the spray-drying step.

The drying gas may be any gas. Preferably, the gas is air or an inert gas such as nitrogen, nitrogen-enriched air or argon. The temperature of the drying gas at the gas inlet of the spray-drying chamber is typically from about 60° C. to about 300° C.

A typical spray-drying apparatus comprises a spray-drying chamber, atomizing means for introducing the feed mixture into the spray-drying chamber in the form of droplets, a source of heated drying gas that flows into the spray-drying chamber through an inlet, and an outlet for the heated drying gas. The spray-drying apparatus also comprises a means for collecting the solid pharmaceutical powder that is produced.

The atomizing can be a rotary atomizer, a pneumatic nozzle, or a high pressure nozzle. A preferred atomizer is the high pressure nozzle where liquid feed is pumped to the nozzle under pressure. Pressure energy is converted to kinetic energy, and feed issues from the nozzle orifice as a high-speed film that readily disintegrates into a spray as the film is unstable. The feed is made to rotate within the nozzle using a swirl insert or swirl chamber resulting in cone shaped spray patterns emerging from the nozzle orifice. Swirl insert, swirl chamber and orifice dimensions together with variation of pressure gives control over feed rate and spray characteristics. The size of the droplets produced by high pressure nozzles depends on the operating parameters and can be in the range from about 5 to 125 μm, in particular from about 20 to 50 μm.

Optionally, further excipients may be included in the feed mixture. Such excipients may be included in order to improve properties of the feed mixture or the resulting solid pharmaceutical composition, such as handling or processing properties. Regardless of whether or not excipients are added to the feed mixture, which obviously results in them being incorporated in the spray-dried solid dispersion, excipients may also be mixed with the resulting solid spray-dried dispersion during formulation into a desired dosage form. The spray-dried solid dispersion may be subjected to further processing steps depending on the nature of the final dosage form. For example, the pharmaceutical composition may be subjected to a post-drying process, or may undergo slugging or roller compacting prior to tabletting. To improve the strength of the resulting tablets, a sufficient amount of a binder can be added to the mixture that is compressed.

The spray-dried solid dispersion may be formulated into a pharmaceutical formulation. The latter comprises the spray-dried solid dispersion produced by the process of the invention and a carrier, which may comprise one or more pharmaceutically acceptable excipients. The latter include surfactants, solubilizers, disintegrants, pigments, flavoring agents, fillers, lubricants, glidants, preservatives, thickening agents, buffering agents and pH modifiers. Typical surfactants include sodium lauryl sulphate, Cremophor RH 40, Vitamin E TPGS and polysorbates, such as Tween 20™. Typical pH modifiers are acids, such as citric acid or succinic acid, bases or buffers.

The dosage form may be coated using standard coating materials and procedures. A coating that may be used is Opadry™.

The administration of a dosage form in accordance with the present invention may suffice to treat HIV infection although it may be recommendable to co-administer other HIV inhibitors. The latter preferably include HIV inhibitors of other classes, in particular one or preferably two NRTIs, but also a fusion inhibitor can be added. HIV inhibitors that may be co-administered by preference are those used in HAART combinations.

In certain instances, the treatment of HIV infection may be limited to only the dosage form of the invention, without co-administration of further HIV inhibitors. This option may be recommended, for example, where the viral load is relatively low, e.g. where the viral load (represented as the number of copies of viral RNA in a specified volume of serum) is below about 200 copies/ml, in particular below about 100 copies/ml, more in particular below 50 copies/ml, specifically below the detection limit of the virus. This type of therapy may be applied after initial treatment with a combination of HIV drugs, such as any of the HAART combinations during a certain period of time until the viral load in blood plasma reaches the afore mentioned low viral level.

In a further aspect, the present invention relates to the use of a dosage form in accordance with the invention, for the manufacture of a medicament for maintenance therapy of a subject infected with HIV. The present invention also relates to the use of a dosage form in accordance with the invention, for the manufacture of a medicament for treating a subject infected with HIV, wherein the dosage form is combined with two different NRTIs.

As used herein the term “treatment of HIV infection” relates to a situation of the treatment of a subject being infected with HIV. The term “subject” in particular relates to a human being.

The doses of TMC125 and TMC114 in the dosage forms of the invention are selected so as to keep the blood plasma concentration of these anti-HIV agents above the minimum blood plasma level between two administrations. The term “minimum blood plasma level” in this context refers to the lowest efficacious blood plasma level, the latter being that blood plasma level of both actives that provides effective treatment of HIV. The plasma levels of these anti-HIV compounds should be kept above these threshold blood plasma levels because at lower levels the drugs may no longer be effective thereby increasing the risk of mutations.

The dosage forms of the present invention provide effective treatment of HIV infection in that the viral load is reduced while keeping viral replication suppressed. The limited number of drug administrations adds to the patients' compliance with the prescribed therapy.

As used herein, the word “substantially” does not exclude “completely” e.g. a composition which is “substantially free” from Y may be completely free from Y.

Where necessary, the word “substantially” may be omitted from the definitions describing the invention. The term “about” in connection with a numerical value is meant to have its usual meaning in the context of the numerical value. Where necessary the word “about” may be replaced by the numerical value ±10%, or ±5%, or ±2%, or ±1%.

All documents cited herein are incorporated by reference in their entirety.

### EXAMPLES

### Example 1

### 1) Manufacturing of Spray-Dried Powders

Feed mixtures for the spray-dried formulations were prepared by dissolving TMC125 and the polymer in the solvent and adding microcrystalline cellulose. The polymer-type, solvent and the amounts of the components used are listed under feed mixture (iv) mentioned hereinabove. The feed mixture was then admitted to an SD-12.5-N, closed cycle spray-drying chamber via a high-pressure nozzle in co-current mode. The resulting solid pharmaceutical composition was collected from the cyclone, post-dried under vacuum at elevated temperature to decrease the residual solvent level. The dried powder was sieved and the powder fraction with a particle size between 45 and 100 micron was retained.

### 2) Manufacturing of Combination Tablets

TABLE 1
composition of the combination tablets

| | Formulation 1 | Formulation 2 |
|---|---|---|
| Ingredient Name | mg/tablet | mg/tablet |
| TMC125 | 100.0 | 100.0 |
| HPMC 2910 5 cps | 300.0 | 300.0 |
| MCC | 50.0 | 50.0 |
| Crosscarmellose sodium | 20.0 | 20.0 |
| TMC114 | 325.23 | 325.23 |
| Colloidal anhydrous silica | 5.0 | 5.0 |
| Crosscarmellose sodium | 50.0 | 50.0 |
| MCC (Ceolus KG802 ™) | 144.77 | 50.0 |
| Silicified MCC | | 194.77 |
| Magnesium stearate | 5.0 | 5.0 |
| Total | 1000.0 | 1100.0 |

A mixture of spray-dried TMC125 in HPMC with microcrystalline cellulose (MCC), Crosscarmellose sodium (Ac-Di-Sol™) and TMC114 was slugged and the obtained tablets were broken and sieved over a 850μ stainless steel sieve. The rest of the external phase ingredients Colloidal anhydrous silica, Crosscarmellose sodium (Ac-Di-Sol™) MCC and Magnesium stearate were sieved, added and mixed for 5 minutes using a Turbula™ mixer. The tablets were compressed on an excentric press (Courtoy AC27™)

The tablets were measured on dissolution in a medium of 0.01M HCl+1% sodium laurylsulfate (SLS), 50 rpm. The results reported in the following tables are the percentages of release drug substance. Exp 1, exp 2, etc. refer to a first, a second, etc. experiment under the same conditions.

TABLE 2
Dissolution of TMC125 out of combination tablet
of Formula 1, obtained with slugging.

| | time (min) | | | | | | | | |
|---|---|---|---|---|---|---|---|---|---|
| | 5 | 10 | 15 | 20 | 30 | 45 | 60 | 120 | 180 |
| exp 1 | 42.13 | 57.36 | 65.87 | 71.43 | 78.55 | 84.65 | 88.07 | 93.89 | 96.01 |
| exp 2 | 42.16 | 57.61 | 66.14 | 71.81 | 78.89 | 85.09 | 88.32 | 93.69 | 95.53 |
| exp 2 | 40.98 | 56.96 | 65.80 | 71.72 | 78.78 | 85.17 | 88.40 | 94.05 | 96.05 |

TABLE 3
Dissolution of TMC114 out of combination tablet
of Formula 1, obtained with slugging.

| | time (min) | | | | | | | | |
|---|---|---|---|---|---|---|---|---|---|
| | 5 | 10 | 15 | 20 | 30 | 45 | 60 | 120 | 180 |
| exp 1 | 85.70 | 93.15 | 94.75 | 95.68 | 96.22 | 96.95 | 96.91 | 96.92 | 96.92 |
| exp 2 | 86.02 | 92.73 | 94.15 | 94.87 | 95.67 | 96.10 | 95.93 | 95.81 | 95.77 |
| exp 3 | 85.43 | 92.79 | 94.87 | 95.71 | 96.23 | 96.90 | 96.63 | 97.39 | 97.41 |

In the above tables, results of three experiments are listed.

### Example 2

### 1) Manufacturing of Spray-Dried Powders

Spray-drying of TMC125 was performed as described in Example 1.

### 2) Manufacturing of the Bulk Blend for Roller Compaction

TABLE 4

| component in mg/tablet | exp. 1 | exp. 2 |
|---|---|---|
| Spray dried TMC125 | 450.0 | 450.0 |
| TMC114.EtOH | 325.23 | 325.23 |
| Croscarmellose Sodium | 20.0 | 20.0 |
| Total | 795.23 | 795.23 |

The ingredients were sieved manually over a stainless steel sieve of 1 mm, and then blended in a 1001 Gallay tumble blender at 10 rpm for 10 minutes. Roller compaction was performed on a Gerteis Polygran™ 250/100/3 roller compactor.

TABLE 5
Roller compaction settings.

| | exp. 1 | exp. 2 |
|---|---|---|
| Type of roller | smooth | smooth |
| Diameter of the roller (mm) | 250 | 250 |
| Width roller (mm) | 100 | 100 |
| Force (kN/cm) | 5 | 12 |
| Sieve (mm) | 0.8 | 0.8 |

TABLE 6
Results of the roller compacted material

| | exp. 1 | exp. 2 |
|---|---|---|
| Mean force (kN/cm) | 5 | 12 |
| Mean gap (mm) | 2.99 | 2.98 |
| Yield (%) | 97.0 | 98.33 |
| Bulk volume (ml/g) | 2.68 | 2.00 |
| Tapped volume (ml/g) | 2.14 | 1.64 |
| D50 (μm) | 170 | 328 |

TABLE 7
Final blend and compression

| Material in mg/tablet | exp. 1 | exp. 2 |
|---|---|---|
| TMC125 | 100.00 | 100.00 |
| HPMC 2910 5 cps | 300.00 | 300.00 |
| MCC | 50.00 | 50.00 |
| Croscarmellose Sodium | 20.00 | 20.00 |
| TMC114.EtOH | 325.23 | 325.23 |
| total | 795.23 | 795.23 |
| Croscarmellose Sodium | 50.00 | 50.00 |
| Colloidal anhydrous | 5.00 | 5.00 |
| silica | | |
| MCC | 144.77 | 144.77 |
| Magnesium Stearate | 5.00 | 5.00 |
| total | 1000.00 | 1000.00 |

All ingredients except for Magnesium Stearate were sieved over a stainless steel sieve of 1 mm, and then blended in a 1001 Gallay™ tumble blender at 10 rpm for 10 minutes. Then sieved magnesium stearate was added to the blend and mixed for another 4 minutes using the same speed.

The trials were performed on a 16 punch rotary press.

A compression profile study was done to evaluate tablet properties using different compression forces.

TABLE 8
Dissolution of TMC125 as a function of roller compactor force (5 kN)
Dissolution of TMC125 out of combination tablet Exp 1

| | time (min) | | | | | | | | | |
|---|---|---|---|---|---|---|---|---|---|---|
| | 5 | 10 | 15 | 20 | 30 | 45 | 60 | 120 | 180 | 210 |
| 752 daN | 26.16 | 43.32 | 54.92 | 70.36 | 74.65 | 85.02 | 90.48 | 97.02 | 99.37 | 100.46 |
| 1030 daN | 31.67 | 46.35 | 57.37 | 65.95 | 75.10 | 80.93 | 84.83 | 93.07 | 96.29 | 98.15 |
| 1235 daN | 36.99 | 49.05 | 58.16 | 66.83 | 70.36 | 79.61 | 83.13 | 91.99 | 93.65 | 99.53 |
| 1537 daN | 26.92 | 39.81 | 46.56 | 55.60 | 61.30 | 69.41 | 73.62 | 84.68 | 87.15 | 96.11 |
| 1775 daN | 20.24 | 31.01 | 37.58 | 44.30 | 50.30 | 56.99 | 63.87 | 75.28 | 76.78 | 89.97 |

TABLE 9
Dissolution of TMC125 as a function of roller compactor force (5 kN)
Dissolution of TMC114 out of combination tablet Exp 1

| | time (min) | | | | | | | | | |
|---|---|---|---|---|---|---|---|---|---|---|
| | 5 | 10 | 15 | 20 | 30 | 45 | 60 | 120 | 180 | 210 |
| 752 daN | 34.92 | 53.74 | 65.16 | 79.03 | 83.31 | 91.67 | 95.20 | 97.26 | 97.32 | 97.16 |
| 1030 daN | 50.89 | 66.00 | 76.00 | 83.73 | 90.48 | 93.51 | 94.87 | 96.66 | 96.42 | 96.37 |
| 1235 daN | 74.74 | 78.53 | 83.86 | 90.22 | 61.29 | 93.03 | 93.16 | 95.06 | 93.31 | 97.12 |
| 1537 daN | 63.88 | 74.30 | 76.94 | 85.43 | 85.70 | 89.43 | 90.04 | 93.30 | 91.15 | 97.34 |
| 1775 daN | 49.61 | 59.09 | 63.53 | 69.50 | 72.04 | 75.32 | 80.05 | 85.20 | 82.66 | 94.52 |

TABLE 10
Dissolution of TMC125 as a function of roller compactor force (12 kN)
Dissolution of TMC125 out of combination tablet Exp 2

| | time (min) | | | | | | | | | |
|---|---|---|---|---|---|---|---|---|---|---|
| | 5 | 10 | 15 | 20 | 30 | 45 | 60 | 120 | 180 | 210 |
| 759 daN | 25.92 | 39.80 | 48.13 | 54.99 | 64.23 | 72.02 | 76.80 | 86.94 | 91.85 | 94.65 |
| 1051 daN | | 35.84 | 42.58 | 49.61 | 56.96 | 62.98 | 70.28 | 81.14 | 87.60 | 92.40 |
| 1266 daN | | 14.60 | 20.96 | 28.70 | 42.77 | 52.56 | 61.74 | 76.12 | 81.10 | 88.90 |
| 1496 daN | | 10.87 | 13.21 | 16.71 | 24.57 | 36.59 | 45.84 | 66.54 | 79.12 | 82.30 |

TABLE 11
Dissolution of TMC114 as a function of roller compactor force (12 kN)
Dissolution of TMC114 out of combination tablet Exp 2

| | time (min) | | | | | | | | | |
|---|---|---|---|---|---|---|---|---|---|---|
| | 5 | 10 | 15 | 20 | 30 | 45 | 60 | 120 | 180 | 210 |
| 759 daN | 40.58 | 58.53 | 67.90 | 75.48 | 84.13 | 89.98 | 92.76 | 96.07 | 96.42 | 96.50 |
| 1051 daN | | 57.60 | 65.07 | 73.32 | 79.89 | 83.59 | 89.25 | 92.98 | 94.23 | 95.85 |
| 1266 daN | | 21.67 | 30.69 | 42.99 | 63.22 | 73.73 | 83.02 | 91.42 | 90.96 | 95.69 |
| 1496 daN | | 15.57 | 18.44 | 23.37 | 34.54 | 50.91 | 62.57 | 82.75 | 91.76 | 91.31 |

The higher the compression force, the slower is the dissolution of the active ingredient. The dissolution of TMC125 appears to be more influenced by the compression force than the dissolution of TMC114.

### Example 3

### Coating

TABLE 12
Dissolution of TMC125 coated and uncoated
Dissolution of TMC125 out of combination tablet Exp 2

| | time (min) | | | | | | | | | |
|---|---|---|---|---|---|---|---|---|---|---|
| | 5 | 10 | 15 | 20 | 30 | 45 | 60 | 120 | 180 | 210 |
| 1030 daN uncoated | 31.67 | 46.35 | 57.37 | 65.95 | 75.10 | 80.93 | 84.83 | 93.07 | 96.29 | 98.15 |
| 1266 daN uncoated | | 14.60 | 20.96 | 28.70 | 42.77 | 52.56 | 61.74 | 76.12 | 81.10 | 88.90 |
| 1030 daN coated | 16.77 | 27.45 | 37.80 | 46.96 | 62.47 | 80.84 | 89.56 | 98.21 | 99.61 | 99.34 |
| 1266 daN coated | 21.99 | 37.79 | 46.23 | 52.07 | 60.11 | 67.31 | 72.54 | 83.87 | 88.91 | 91.43 |

TABLE 13
Dissolution of TMC114 coated and uncoated
Dissolution of TMC114 out of combination tablet Exp 1

| | time (min) | | | | | | | | | |
|---|---|---|---|---|---|---|---|---|---|---|
| | 5 | 10 | 15 | 20 | 30 | 45 | 60 | 120 | 180 | 210 |
| 1030 daN uncoated | 50.89 | 66.00 | 76.00 | 83.73 | 90.48 | 93.51 | 94.87 | 96.66 | 96.42 | 96.37 |
| 1266 daN uncoated | | 21.67 | 30.69 | 42.99 | 63.22 | 73.73 | 83.02 | 91.42 | 90.96 | 95.69 |
| 1030 daN coated | 21.27 | 30.50 | 39.44 | 50.01 | 64.88 | 82.58 | 90.14 | 95.10 | 95.90 | 98.78 |
| 1266 daN coated | 41.89 | 63.55 | 71.90 | 77.52 | 84.07 | 87.73 | 90.31 | 93.28 | 93.67 | 91.44 |

TABLE 14
Formulation 2 tablet with total weight of 1100 mg
2 phase method/75 rpm

| product | 5 | 10 | 15 | 20 | 30 | 45 | 60 | 120 | 180 | 210 |
|---|---|---|---|---|---|---|---|---|---|---|
| TMC125 | 45 | 56 | 62 | 66 | 72 | 78 | 82 | 90 | 93 | 94 |
| uncoated | | | | | | | | | | |
| TMC125 coated | 49 | 60 | 67 | 71 | 77 | 83 | 87 | 94 | 96 | 97 |
| TMC114 | 78 | 85 | 88 | 91 | 94 | 96 | 97 | 97 | 97 | 97 |
| uncoated | | | | | | | | | | |
| TMC114 coated | 82 | 89 | 93 | 94 | 97 | 98 | 98 | 98 | 98 | 98 |

Two coating trials were performed using the tablet core samples with a compression force of 1030 daN and 1266 daN. The coating material was polyvinyl alcohol (PVA). The film coated tablets were measured on dissolution in medium 0.01 M HCl+1% SLS, 50 rpm.

The release of TMC125 was somewhat faster out of the coated tablets compared to the cores. There was only a minor impact of the coating on the release of TMC114.

1. A solid pharmaceutical oral dosage form comprising:
(a) from about 15 mg to about 200 mg of TMC125, or from about 25 mg to about 150 mg of TMC125, or from about 50 to about 150 mg of TMC125, or from about 80 to about 120 mg of TMC125, dispersed in a solid dispersion with a water-soluble polymer;
(b) from about 50 to about 600 mg, or from about 50 mg to about 500 mg of free-form equivalent TMC114; or from about 250 mg to about 450 mg of free-form equivalent TMC114, or from about 250 mg to about 350 mg of free-form equivalent of TMC114;
(c) from about 200 mg to about 400 mg of a carrier;
the total weight of the dosage form not exceeding 1300 mg.

2. The dosage form according to claim 1, comprising:
(a) from about 80 to about 120 mg of TMC125, dispersed in a solid dispersion with a water-soluble polymer;
(b) from about 250 mg to about 350 mg of free-form equivalent of TMC114;
(c) from about 200 mg to about 400 mg of a carrier;
the total weight of the dosage form not exceeding 1300 mg.

3. The dosage form according to claim 1, comprising
(a) about 100 mg of TMC125 dispersed in a solid dispersion with a water-soluble polymer;
(b) about 300 mg free-form equivalent of TMC114, or in particular about 325 mg of TMC114 monoethanolate; or about 400 mg free-form equivalent of TMC114, or in particular about 434 mg of TMC114 monoethanolate;
(c) from about 200 mg to about 400 mg of a carrier;
the total weight of the dosage form not exceeding 1300 mg.

4. The dosage form according to claim 1, wherein the TMC125 is present as the free form and the TMC114 as the monoethanolate form.

5. The dosage form according to claim 1, wherein the total weight of the dosage form does not exceed 1100 mg.

6. The dosage form according to claim 1, wherein the TMC125 is dispersed in from about 100 to about 500 mg of a water-soluble polymer.

7. The dosage form according to claim 1, wherein the TMC125 is dispersed in from about 200 to about 400 mg of a water-soluble polymer.

8. The dosage form according to claim 1, wherein the TMC125 is dispersed in about 300 mg of a water-soluble polymer.

9. The dosage form according to claim 1, wherein the solid dispersion of TMC125 comprises from about 10 mg to about 100 mg, or from about 20 mg to about 80 mg, or from about 30 mg to about 70 mg, or from about 40 mg to about 60 mg, or about 50 mg, of microcrystalline cellulose.

10. The dosage form according to claim 1, wherein the weight/weight ratio between the TMC125 and the water-soluble polymer is in the range from about 1:1 to about 1:5.

11. The dosage form according to claim 1, wherein the weight/weight ratio between the TMC125 and the water-soluble polymer is about 1:3.

12. The dosage form according to claim 1, wherein the water-soluble polymer is HPMC.

13. The dosage form according to claim 2, wherein the TMC125 is present as the free form and the TMC114 as the monoethanolate form.

14. The dosage form according to claim 3, wherein the TMC125 is present as the free form and the TMC114 as the monoethanolate form.

15. The dosage form according to claim 2, wherein the total weight of the dosage form does not exceed 1100 mg.

16. The dosage form according to claim 3, wherein the total weight of the dosage form does not exceed 1100 mg.

17. The dosage form according to claim 4, wherein the total weight of the dosage form does not exceed 1100 mg.

18. The dosage form according to claim 2, wherein the TMC125 is dispersed in about 300 mg of a water-soluble polymer.

19. The dosage form according to claim 3, wherein the TMC125 is dispersed in about 300 mg of a water-soluble polymer.

20. The dosage form according to claim 4, wherein the TMC125 is dispersed in about 300 mg of a water-soluble polymer.
